(19) United States
(12) Patent Application Publication
(10) Pub. No.: US 2022/0240552 A1
(43) Pub. Date: Aug. 4, 2022

(54) HARMFUL-TO-HEALTH SUBSTANCE REMOVING AGENT AND HEALTH FOOD

(71) Applicant: DEXERIALS CORPORATION, Shimotsuke-shi, Tochigi (JP)

(72) Inventor: Mayu INOMATA, Tochigi (JP)

(73) Assignee: DEXERIALS CORPORATION, Shimotsuke-shi, Tochigi (JP)

(21) Appl. No.: 17/597,576

(22) PCT Filed: Jun. 23, 2020
(86) PCT No.: PCT/JP2020/024522

§ 371 Date: Jan. 12, 2022

(30) Foreign Application Priority Data
Jul. 23, 2019 (JP) 2019-135480

(51) Int. Cl. A23L 5/20; A23L 33/105; A23L 29/00; B01J 20/20; B01J 20/28

(52) U.S. Cl. CPC A23L 5/273; A23L 33/105; B01J 20/28083; B01J 20/20; B01J 20/28071; A23L 29/015

(57) ABSTRACT

A harmful-to-health substance removing agent includes a plant-derived porous carbon material having a mesopore volume of 0.10 cm³/g or greater.

### TECHNICAL FIELD

The present invention relates to a harmful-to-health substrate removing agent and a health food.

### BACKGROUND ART

Hitherto, many cleansing agents formulated with charcoal, activated carbon, and medicinal carbon have been commercially available. Some of these products emphasize a body odor removing effect or an antibacterial effect in addition to a high cleansing effect, and feature a high safety.

However, the charcoal, activated carbon, and medicinal carbon have a weak adsorbability for having harmful substances adsorb thereto because of insufficient growth of mesopores therein. Therefore, it is necessary to apply them in a large amount, which is highly burdening to the user. Particularly, when the medicinal carbon is applied in a large amount, side effects such as constipation occur.

Hence, an adsorbent using a plant-derived porous carbon material has been proposed (for example, see PTL 1).

### CITATION LIST

### Patent Literature

PTL 1: Japanese Patent No. 5168240

### SUMMARY OF INVENTION

### Technical Problem

However, PTL 1 neither describes nor suggests a use intended to safely and quickly remove a harmful-to-health substance that may damage the human health and a specific rate of removal of a harmful-to-health substance.

The present invention aims for solving the various problems in the related art and achieving an object described below. That is, the present invention has an object to provide a harmful-to-health substance removing agent that can quickly remove a harmful-to-health substance such as advanced glycation end products (AGEs), lipids, histamine, and edible tar dyes, and a health food.

### Solution to Problem

Means for solving the above problems are as follows.

<1> A harmful-to-health substance removing agent, including:

a plant-derived porous carbon material having a mesopore volume of 0.10 cm³/g or greater.

<2> The harmful-to-health substance removing agent according to <1>,

wherein the mesopore volume of the porous carbon material is 0.15 cm³/g or greater.

<3> The harmful-to-health substance removing agent according to <1> or <2>,

wherein the mesopore volume of the porous carbon material is greater than a micropore volume.

<4> The harmful-to-health substance removing agent according to any one of <1> to <3>,

wherein the porous carbon material has a median diameter of 1 micrometer or greater but 200 micrometers or less.

<5> The harmful-to-health substance removing agent according to any one of <1> to <4>,

wherein a raw material of the plant-derived porous carbon material is chaff of rice, barley, wheat, rye, Japanese barnyard millet, or millet.

<6> The harmful-to-health substance removing agent according to <5>,

wherein the raw material of the plant-derived porous carbon material is chaff of rice.

<7> The harmful-to-health substance removing agent according to any one of <1> to <6>,

wherein a harmful-to-health substance is an advanced glycation end product.

<8> The harmful-to-health substance removing agent according to <7>,

wherein a rate of removal of the advanced glycation end product is 90% or higher.

<9> The harmful-to-health substance removing agent according to any one of <1> to <6>,

wherein a harmful-to-health substance is histamine.

<10> The harmful-to-health substance removing agent according to <9>,

wherein a rate of removal of the histamine is 90% or higher.

<11> The harmful-to-health substance removing agent according to any one of <1> to <6>,

wherein a harmful-to-health substance is a lipid.

<12> The harmful-to-health substance removing agent according to <11>,

wherein an amount of the lipid adsorbed per 1 g of the porous carbon material is 1.0 g or greater.

<13> The harmful-to-health substance removing agent according to any one of <1> to <6>,

wherein a harmful-to-health substance is an edible tar dye.

<14> The harmful-to-health substance removing agent according to <13>,

wherein a rate of removal of the edible tar dye is 90% or higher.

<15> A health food, including

the harmful-to-health substance removing agent according to any one of <1> to <14>.

### Advantageous Effects of Invention

The present invention can solve the various problems in the related art, achieve the object described above, and provide a harmful-to-health substance removing agent that can quickly remove a harmful-to-health substance such as advanced glycation end products (AGEs), lipids, histamine, and edible tar dyes, and a health food.

### BRIEF DESCRIPTION OF DRAWINGS

FIG. 1 is diagram indicating the results of an adsorption test on advanced glycation end products (AGEs) in Example 1;

FIG. 2 is a diagram indicating the results of an adsorption test on Red No. 102, which is an edible tar dye, in Example 4;

FIG. 3 is a diagram indicating the results of an adsorption test on Blue No. 1, which is an edible tar dye, in Example 4; and

FIG. 4 is a diagram indicating the results of an adsorption test on Yellow No. 4, which is an edible tar dye, in Example 4.

### DESCRIPTION OF EMBODIMENTS

### (Harmful-to-Health Substance Removing Agent)

A harmful-to-health substance removing agent of the present invention contains a plant-derived porous carbon material having a mesopore volume of 0.10 cm³/g or greater, and further contains other components as needed.

The harmful-to-health substance removing agent of the present invention is a plant-derived porous carbon material and includes more mesopores (i.e., has a greater mesopore volume) than other activated carbon and carbon materials. Therefore, the harmful-to-health substance removing agent has a high adsorption amount of a harmful-to-health substance and a high adsorption speed of a harmful-to-health substance, and can efficiently remove a harmful-to-health substance even when ingested in a small amount.

Not only the porous carbon material, examples of the harmful-to-health substance removing agent also include activated carbon and plant charcoal powder pigments so long as activated carbon and plant charcoal powder pigments have a high mesopore volume and can work the effect of the present invention.

### <Porous Carbon Material>

### —Mesopore Volume—

The porous carbon material is a plant-derived material, and the mesopore volume of the porous carbon material is 0.10 cm³/g or greater, preferably 0.15 cm³/g or greater, and more preferably 0.15 cm³/g or greater but 0.5 cm³/g or less. When the mesopore volume is less than 0.1 cm³/g, mesopores have not grown enough, and advantages such as excellent adsorption of large molecules and an excellent high-speed adsorbability cannot be obtained. On the other hand, when the mesopore volumes is extremely high, it becomes harder to obtain a high bulk specific gravity.

The porous carbon material contains many pores. Pores are classified into mesopores, micropores, and macropores. Here, mesopores are defined as pores having a pore diameter of from 2 nm through 50 nm, micropores are defined as pores having a pore diameter of less than 2 nm, and macropores are defined as pores having a pore diameter of greater than 50 nm.

The mesopore volume can be measured with, for example, the instrument described below.

Based on measurement of a nitrogen adsorption isotherm with 3FLEX available from Micromeritics Japan, G.K., the mesopore volume can be calculated by a BJH method.

The BJH method is a method widely used as a pore distribution analyzing method. In a pore distribution analysis by the BJH method, first, nitrogen as adsorbing molecules is adsorbed to and desorbed from the adsorbent (porous carbon material), to thereby obtain a desorption isotherm. Then, based on the obtained desorption isotherm, the thickness of an adsorbing layer when the adsorbing molecules gradually adsorb and desorb from a state where the pores are filled with the adsorbing molecules (e.g., nitrogen), and the inner diameter (double a core radius) of pores generated as a result are obtained. The pore radius rₚ is calculated according to the formula (1), and the pore volume is calculated according to the formula (2). Then, based on the pore radius and the pore volume, a pore volume change ratio (dVₚ/drₚ) with respect to a pore diameter (2rₚ) is plotted. In this way, a pore distribution curve is obtained (see Manual of BELSORP-MINI and BELSORP analyzing software available from Bel Japan Inc., pp. 85-88).

rₚ=t+rₖ (1)

Vₚₙ=Rₙ*dVₙ−Rₙ·dtₙ·c·ΣAₚᵢ (2)

where Rₙ=rₚₙ²/(rₖₙ₋₁+dtₙ)² (3)

In the formulae above, the signs represent the following.

rₚ: pore radius

rₖ: core radius (inner diameter/2) when an adsorbing layer having a thickness of t adsorbs to the inner wall of a pore having a pore radius of rₚ at a pressure concerned

Vₚₙ: pore volume when the n-th adsorption and desorption of nitrogen occurs

dVₙ: amount of change at that time

dtₙ: amount of change of the thickness tₙ of the adsorbing layer when the n-th adsorption and desorption of nitrogen occurs

rₖₙ: core radius at that time

c: fixed value

rₚₙ: pore radius when the n-th adsorption and desorption of nitrogen occurs

ΣAₚⱼ: integrated value of the area of the wall surface of a pore from j=1 to j=n−1

### [Specific Measuring Method]

The porous carbon material (30 mg) is prepared, and the mesopore volume thereof can be measured with 3FLEX set to a condition for measuring a relative pressure (P/PO) range of from 0.0000001 through 0.995.

### —Micropore Volume—

It is preferable that the mesopore volume of the porous carbon material be greater than the micropore volume. When the mesopore volume is greater than the micropore volume, the effect of removing a harmful-to-health substance is good.

The micropore volume is preferably 0.05 cm³/g or greater, and more preferably 0.1 cm³/g or greater but 0.4 cm³/g or less.

The micropore volume can be measured in the same manner as measuring the mesopore volume.

### —Median Diameter—

The median diameter of the porous carbon material is preferably 1 micrometer or greater but 200 micrometers or less, more preferably 1 micrometer or greater but 150 micrometers or less, yet more preferably 5 micrometers or greater but 150 micrometers or less, and particularly preferably 10 micrometers or greater but 100 micrometers or less. When the median diameter is 1 micrometer or greater but 200 micrometers or less, the effect of removing a harmful-to-health substance is good.

The particle diameter can be obtained with, for example, a laser diffraction/scattering particle diameter distribution analyzer LA-950 (available from HORIBA, Ltd.). With LA-950, the particle diameter distribution is measured in a particle diameter range of from 0.01 micrometers through 3,000 micrometers by a wet method. The particle diameter means a particle diameter (median diameter) corresponding to the distribution median of a particle diameter distribution plotting the particle diameter on the horizontal axis and the number frequency on the vertical axis.

### —Bulk Specific Gravity—

The bulk specific gravity of the porous carbon material is preferably 0.15 g/cm³ or greater, more preferably 0.20 g/cm³ or greater but 0.40 g/cm³ or less, and yet more preferably 0.20 g/cm³ or greater but 0.35 g/cm³ or less.

A porous carbon material having grown mesopores (i.e., having a mesopore volume of 0.10 cm³/g or greater) typically has a bulk specific gravity of about 0.10 g/cm³. Therefore, on a per volume basis, the porous carbon material cannot exert advantages such as excellent adsorption of large molecules and an excellent high-speed adsorbability. On the other hand, when the bulk specific gravity of the porous carbon material is 0.15 g/cm³ or greater, the porous carbon material can exert advantages such as excellent adsorption of large molecules and an excellent high-speed adsorbability, both on a per volume basis and a per weight basis.

The bulk specific gravity is a specific gravity (mass per unit volume) obtained by dividing the mass of a powder, which has been brought to have a predetermined shape by, for example, filling a container having a certain volume with the powder by letting the powder freely fall into the container, by the volume of the powder in that shape. A substance having a lower bulk specific gravity is more bulky.

### <Raw Material of Porous Carbon Material>

The raw material of the porous carbon material is preferably a plant-derived material. With a plant-derived material, it is easy to adjust the mesopore volume to the desired value described above. A plant-derived material is advantageous also because a plant-derived material has a low environmental impact.

The plant-derived material is not particularly limited and may be appropriately selected depending on the intended purpose. Examples of the plant-derived material include: chaff and straw of, for example, rice (paddy), barley, wheat, rye, Japanese barnyard millet, and millet; sawdust and wood chips of, for example, cedar, pine, live oak, and oak; and reed, wakame stem, vascular plants vegetating on land, pteridophyte, bryophyte, algae, and seagrass. One of these plant-derived materials may be used alone or two or more of these plant-derived materials may be used in combination. Among these plant-derived materials, chaff of rice is preferable because of a high mesopore volume.

The shape and form of the plant-derived material are not particularly limited. For example, chaff and straw are suitable, or dried products thereof are also suitable. Moreover, materials having subjected to various processes such as fermentation, roasting, and extraction during food and beverage processing for, for example, beer and western liquor can also be used. Particularly, from the viewpoint of proactively recycling industrial wastes, it is preferable to use straw and chaff obtained through processing such as threshing. It is possible to procure large quantities of such straw and chaff obtained through processing, easily from, for example, agricultural cooperatives, liquor companies, and food companies.

The method for producing the porous carbon material is not particularly limited and may be appropriately selected depending on the intended purpose. A method for producing a porous carbon material described below is preferable.

### <Method for Producing Porous Carbon Material>

A method for producing a porous carbon material includes a molding producing step, a carbide producing step, and an activating step, preferably includes a decalcifying step, and further includes other steps as needed.

The method for producing a porous carbon material is a method for producing the porous carbon material of the present invention.

### <Molding Producing Step>

The molding producing step is not particularly limited and may be appropriately selected depending on the intended purpose so long as it is a step of pressure-molding a plant-derived material to obtain a molding.

The plant-derived material is not particularly limited and may be appropriately selected depending on the intended purpose. Examples of the plant-derived material include the plant-derived materials raised as examples in the description of the porous carbon material. Among these plant-derived materials, chaff is preferable because of a high mesopore volume.

The shape of the molding is not particularly limited and may be appropriately selected depending on the intended purpose.

The pressure molding is performed with, for example, a pelletizer commonly used for molding biomass, and ground chaff is molded with addition of water in a manner that the water content ratio will be 3% by mass or greater but 30% by mass or less, preferably 5% by mass or greater but 20% by mass or less. Here, the pressure is determined by frictional resistance between the molding dies and the chaff when the chaff is passed through the molding machine. Hence, it is desirable to adjust the amount of water depending on the size of the molding.

In the pressure molding, heat may be generated due to friction. A heating device may further apply heat.

It is inferred that through appropriate adjustment of the amount of water, pressure, and heat, any water-soluble component contained in the plant-derived material is extracted and bonds the powder particles with each other, to form a molding.

Through pressure-molding the plant-derived material, it is possible to obtain a porous carbon material having more grown mesopores than when the plant-derived material is not pressure-molded.

### <Carbide Producing Step>

The carbide producing step is not particularly limited and may be appropriately selected depending on the intended purpose so long as it is a step of carbonizing the molding to obtain a carbide (carbonaceous material).

The carbonization typically means thermally treating an organic substance (a plant-derived material in the present invention) to convert it to a carbonaceous material (for example, see JIS M0104-1984). Examples of the atmosphere for carbonization include an atmosphere from which oxygen is cut off. Specific examples of such an atmosphere include a vacuum atmosphere, inert gas atmospheres such as nitrogen gas and argon gas, and an atmosphere in which the molding is brought into a kind of a steamed state. The temperature elevation rate until the carbonization temperature is reached is 1 degree C./minute or higher, preferably 3 degrees C./minute or higher, and more preferably 5 degrees C./minute or higher under the atmosphere described above. The upper limit of the carbonization time is 10 hours, preferably 7 hours, and more preferably 5 hours, but is not limited to these times. The lower limit of the carbonization time may be a time in which the molding will be carbonized securely.

The temperature of the thermal treatment is, for example, from 300 degrees C. through 1,000 degrees C.

### <Activating Step>

The activating step is not particularly limited and may be appropriately selected depending on the intended purpose so long as it is a step of activating the carbide. Examples of the activating step include a gas activation method and a chemical activation method.

Activation means growing the porous structure of the carbon material to add pores.

The gas activation method is a method of heating the carbide using, for example, oxygen, water vapor, carbon dioxide, and air as an activator under the gas atmosphere described above at, for example, from 700 degrees C. through 1,000 degrees C. for from some tens of minutes through some hours, to thereby grow a fine structure by volatile components and carbon molecules contained in the carbide. The heating temperature may be appropriately selected depending on, for example, the kind of the plant-derived material, and the kind and concentration of the gas, and is preferably from 800 degrees C. through 950 degrees C.

The chemical activation method is a method of activating the carbide using, for example, zinc chloride, iron chloride, calcium phosphate, calcium hydroxide, magnesium carbide, potassium carbide, and sulfuric acid instead of oxygen and water vapor used in the gas activation method, wash the resultant with hydrochloric acid, adjusting pH with an alkaline aqueous solution, and drying the resultant.

### <Decalcifying Step>

The decalcifying step is not particularly limited and may be appropriately selected depending on the intended purpose so long as it is a step of removing any ash content from the carbide. Examples of the decalcifying step include a method of immersing the carbide in an acidic aqueous solution or an alkaline aqueous solution.

Before the decalcifying step, it is preferable to grind the carbide to a size that enables the acidic aqueous solution or the alkaline aqueous solution to easily osmose through the carbide.

An example of the method for producing a porous carbon material will be described below.

The pressure-molded product of chaff is carbonized by heating under a nitrogen current at 500 degrees C. for 5 hours, to obtain a carbide. Subsequently, the carbide (10 g) is put in an alumina crucible, and subjected to temperature elevation up to 1,000 degrees C. at a temperature elevation rate of 5 degrees C./minute under a nitrogen current (10 liters/minute). Then, the resultant is carbonized at 1,000 degrees C. for 5 hours to be converted to a carbonaceous material (porous carbon material precursor), and subsequently cooled to room temperature. During carbonization and cooling, the nitrogen gas is kept flowing. Next, the carbonaceous material is coarsely ground to a size of 1 cm or less at which it is easy to treat the carbonaceous material with an alkali, and any ash content contained in the material is removed using a 1 mol % sodium hydroxide aqueous solution. Subsequently, the material is washed to remove any alkali on the surface of the material, and then further washed. Subsequently, the material is thermally treated at 950 degrees C. under a water vapor atmosphere, to obtain a plant-derived porous carbon material having a high mesopore volume.

The harmful-to-health substance removing agent of the present invention may contain other additives in addition to the plant-derived porous carbon material.

### —Other Additives—

The other additives are not particularly limited and may be appropriately selected depending on the intended purpose. Examples of the other additives include linolenic acid, fish orbital oil, docosahexaenoic acid (DHA), eicosapentaenoic acid (EPA), lactose, sucrose, mannitol, synthetic or natural gums such as corn starch, excipients such as crystalline cellulose, binders such as starch, cellulose derivatives, gum arabic, gelatin, and polyvinyl pyrrolidone, disintegrants such as carboxymethyl cellulose calcium, carboxymethyl cellulose sodium, starch, corn starch, and sodium alginate, lubricants such as talc, magnesium stearate, and sodium stearate, fillers such as calcium carbonate, sodium carbonate, calcium phosphate, and sodium phosphate, diluents, various vitamins, lactic acid bacteria, green juice (green barley extract), sweeteners, proteins (e.g., whey-derived, soybean-derived, egg white-derived, meat-derived, green pea-derived, and brown rice-derived), and minerals. One of these other additives may be used alone or two or more of these other additives may be used in combination.

The ingestion amount of the harmful-to-health substance removing agent is not particularly limited and may be appropriately selected depending on the intended purpose. A suitable amount is from 1 g through 10 g per day for an adult. The ingestion amount may be appropriately increased or decreased depending on, for example, age, body weight, and symptoms.

The method for ingesting the harmful-to-health substance removing agent of the present invention is not particularly limited and may be appropriately selected depending on the intended purpose. Examples of the method include oral administration, parenteral administration, and digestive tract administration. Among these methods, oral administration is preferable.

Examples of the form of the harmful-to-health substance removing agent include tablet, pill, powder, granule, syrup, liquid, suspension, emulsion, and capsule.

It is possible to produce, for example, the tablet by adding additives commonly used and processing the resultant with, sugar coating, gelatin, enteric coating, and film coating commonly used.

### <Harmful-to-Health Substance>

The harmful-to-health substance means all substances that are harmful to the human health. Examples of the harmful-to-health substance include advanced glycation end products (AGEs), lipids, histamine, edible tar dyes, and acrylamides.

### <<Advanced Glycation End Products (AGEs)>>

A reaction between an amino group of amino acids, peptides, and proteins and a reducing sugar such as ketone, aldehyde, and, particularly, glucose, to produce a brown pigment is referred to as Maillard reaction. Products produced as final products of the Maillard reaction are referred to as advanced glycation end products (AGEs).

Advanced glycation end products (AGEs) is a generic term for products of glycation reactions. Advanced glycation end products are substances in which proteins and sugars are bonded with each other, and are said to promote in-vivo glycation and promote oxidation and aging. Besides, advanced glycation end products have been found to have something to do with, for example, dementia, cancers, hypertension, arteriosclerosis, and Alzheimer's disease.

The rate of removal of advanced glycation end products is preferably 90% or higher, and more preferably 95% or higher.

The rate of removal of advanced glycation end products (AGEs) can be obtained in the manner described below.

The rate of removal of advanced glycation end products can be calculated according to Mathematical formula 1 below, where A represents the absorbance, at a maximum absorption wavelength, of an aqueous solution obtained by dissolving alanine (25 g) and glucose (50 g) in water (300 mL), subsequently heating the resultant at 95 degrees C. for 9 hours, naturally cooling the resultant, and subsequently diluting the resultant ten-fold with water, and B represents the absorbance, at the maximum absorption wavelength, of a product obtained by adding the harmful-to-health substance removing agent (porous carbon material) (0.3 g) to the aqueous solution (40 mL) and subsequently stirring the resultant for 5 minutes.

Rate of removal of advanced glycation end products (%)=[(A−B)/A]×100 <Mathematical Formula 1>

### <<Histamine>>

Histamine is an active amine having a molecular formula of C₅H₉N₃ and a molecular weight of 111.14, and is a derivative of histidine, which is a kind of an amino acid.

Red-flesh fish such as tuna, bonito, and mackerel contain a plenty of free histidine. If these fish are managed inappropriately such as being left at normal temperature, bacteria (histamine-producing bacteria) proliferate and produce histamine from free histidine.

If people eat fish containing a plenty of histamine or processed products of such fish, they may develop an allergy-like histamine food poisoning. As histamine is thermally stable, histamine once produced, even if in heated foods such as roasted foods and fried foods, causes food poisoning.

Histamine is also contained in fermented foods such as wine and cheese, not only in fish or processed products of fish.

Domestically, no standard for the histamine concentration in foods has been stipulated, whereas Codex Standard stipulates a standard for the histamine concentration in, for example, canned foods of fish having a high free histidine content. Countries such as European countries, the United States, Canada. Australia, and New Zealand stipulate standards for the histamine concentration in fish and processed products of fish.

The rate of removal of histamine is preferably 90% or higher and more preferably 95% or higher.

The rate of removal of histamine can be obtained in the manner described below.

(1) A histamine aqueous solution is produced by adding histamine (100 mg) into water (500 mL).

(2) The harmful-to-health substance removing agent (porous carbon material) (0.3 g) is added into the histamine aqueous solution (40 mL), and the resultant is stirred for 5 minutes.

(3) The histamine aqueous solution subjected to filtration is caused to develop a color using a commercially available histamine quantification kit (product name “CHECKCOLOR HISTAMINE”, available from Kikkoman Corporation), and the absorbance of the histamine aqueous solution at a wavelength of 473 nm is measured with a visible spectrophotometer (available from JANWAY, 6300).

(4) The histamine concentration in the histamine aqueous solution is calculated in the manner specified in the histamine quantification kit.

(5) Based on the histamine concentrations in the histamine aqueous solution before and after removal, the rate of removal of histamine is calculated according to Mathematical formula 2 below.

Rate of removal of histamine (%)=[(A−B)/A]×100 <Mathematical formula 2>

“A” represents the histamine concentration in the histamine aqueous solution before treatment, and “B” represents the histamine concentration in the histamine aqueous solution after treatment.

### <<Lipids>>

Lipids are one of the three major nutrients, and serve as energy sources. The main component of lipids is fatty acid. Depending on the substance that bonds with the fatty acid, lipids are classified into simple lipids, complex lipids, and derived lipids.

Examples of edible lipids that are liquid lipids (oils) at normal temperature include sesame oils, soybean oils, corn oils, olive oils, and chili oils. Examples of lipids that are solid lipids at normal temperature include lard, tallow, butter, animal foods (meat and fish), eggs, dairy products, cereals, and beans.

Excessive lipid consumption brings about obesity (accumulation of visceral fat and subcutaneous fat). If visceral fat is accumulated, a hormone excreted from the visceral fat acts to reduce insulin sensitivity to bring about hyperglycemia.

Moreover, excessive lipid consumption induces an excessive energy level, increases neutral fat and cholesterols in the blood, and increases the possibility of suffering arteriosclerosis eventually Arteriosclerosis is the cause of development of lifestyle diseases, which are the cause of various diseases.

The amount of lipids adsorbed per 1 g of the porous carbon material is preferably 1.0 g or greater, and more preferably 1.5 g or greater.

The amount of lipids adsorbed per 1 g of the porous carbon material can be measured in the manner described below.

(1) Water (20 g) and lipids (5 g) are put into a cup.

(2) A harmful-to-health substance removing agent (porous carbon material) is added into the resultant in a predetermined amount. (For volume matching, a chaff-derived porous carbon material, which has a low bulk specific gravity, is added in 3 g, and porous carbon materials derived from coconut shell A, coconut shell B. and Japanese red pine are each added in 5 g.)

(3) The total weight is measured.

(4) Five minutes later, any dry (non-water or lipids-adsorbed) porous carbon material in the cup is removed by suctioning.

(5) The weight of the porous carbon material removed by suctioning is measured.

(6) The water and the lipids are sucked up with a syringe, to measure the weight thereof.

(7) The total amount of the remaining cup, porous carbon material, and lipids adsorbed to the porous carbon material is measured.

(8) The weights of the cup and the porous carbon material before the test are subtracted from the total amount obtained in (7), to obtain the adsorption amount of the lipids.

(9) The amount of the lipids adsorbed per 1 g of the porous carbon material is calculated based on the result of (8) and the addition amount of the porous carbon material.

### <<Edible Tar Dyes>>

Examples of edible tar dyes include red No. 102, which is very widely used in Japan but is prohibited from use in the United States, Canada, and European countries. British Food Standards Agency requested food companies to voluntarily restrict use of red No. 102 in 2007, regarding that it is involved in development of attention deficit disorder and hyperactivity disorder.

Moreover, the United States, Canada, and Belgium regard that red No. 102 may be the cause of cancers and allergies, and ban use of red No. 102 in foods.

Examples of edible tar dyes include red No. 102 (Lithol Rubine BCA. Pigment Red 57), blue No. 1 (Brilliant Blue FCF, Acid Blue 9), and yellow No. 4 (Tartrazine, Acid Yellow 23).

The rate of removal of edible tar dyes is preferably 90% or higher and more preferably 95% or higher.

The rate of removal of edible tar dyes can be obtained in the manner described below.

The rate of removal of edible tar dyes can be calculated according to Mathematical formula 3 below, where A represents the absorbance, at a maximum absorption wavelength, of an aqueous solution obtained by adding edible tar dyes (0.1 g) in water (300 mL), and B represents the absorbance, at the maximum absorption wavelength, of a product obtained by adding the harmful-to-health substance removing agent (porous carbon material) (0.3 g) into the aqueous solution (40 mL) and subsequently stirring the resultant for 5 minutes.

Rate of removal of edible tar dyes (%)=[(A−B)/A]×100 <Mathematical formula 3>

### (Health Food)

A health food of the present invention contains the harmful-to-health substance removing agent of the present invention, and further contains other components as needed.

The health food means a product that has a low risk of doing harm to the human health, and is ingested by oral administration or digestive tract administration in normal social life.

The other components are not particularly limited and may be appropriately selected depending on the intended purpose from auxiliary raw materials or additives or other components used in typical production of foods and beverages. Examples of the other components include glucose, fructose, sucrose, maltose, sorbitol, stevioside, rubusoside, corn syrup, lactose, oligosaccharide, xylitol, trehalose, palatinose, aspartame, acesulfame potassium, sucralose, saccharinates, citric acid, tartaric acid, malic acid, succinic acid, lactic acid, L-ascorbic acid, dl-α-tocopherol, sodium erythorbate, glycerin, propylene glycol, glycerin fatty acid esters, polyglycerin fatty acid esters, sucrose fatty acid esters, sorbitan fatty acid esters, gum arabic, carrageenan, casein, gelatin, pectin, agar, vitamins B, nicotinamide, calcium pantothenate, amino acids, calcium salts, pigments, fragrances, and preservatives. One of these other components may be used alone or two or more of these other components may be used in combination.

The formulation amount of the other components is not particularly limited and may be appropriately selected depending on the intended purpose.

As the form of the health food, any known food or drug form may be employed. As the drug form, for example, powder, capsule, granule, tablet, liquid, and other oral drug forms may be employed. As the typical food form, jelly, syrup, candy, gum, refreshing drink, supplement, and other known food forms may be employed, or a predetermined amount of the health food may be mixed in other known foods.

### EXAMPLES

The present invention will be described below by way of Examples. The present invention should not be construed as being limited to these Examples.

In Examples below, the mesopore volume, micropore volume, median diameter, BET specific surface area, ash content, seepage pH, and bulk specific gravity of the porous carbon material were measured in the manners described below.

### <Mesopore Volume, Micropore Volume, and BET Specific Surface Area>

A porous carbon material (30 mg) was prepared, and the mesopore volume, micropore volume, and BET specific surface area thereof were measured with 3FLEX (obtained from Micromeritics Japan, G.K.) set to a condition for measuring a relative pressure (P/PO) range of from 0.0000001 through 0.995.

### <Median Diameter>

The median diameter was measured with a laser diffraction/scattering particle diameter distribution analyzer LA-950 (obtained from HORIBA, Ltd.).

### <Bulk Specific Gravity>

The bulk specific gravity was a mass per unit volume, and was obtained by dividing the mass of a porous carbon material, which had been brought to have a predetermined shape by filling a container having a certain volume with the porous carbon material by letting the porous carbon material freely fall into the container, by the volume of the porous carbon material in that shape.

### <Ash Content>

A sample was previously dried in a thermostat bath of 115 degrees C.±5 degrees C. for 3 hours, and left to cool to room temperature in a desiccator. The sample was weighed out in an amount of from 1 g through 2 g into a crucible to the digit of 1 mg. The sample was subjected to gradual temperature elevation in an electric furnace (under a temperature elevation setting of 2 hours), and strongly heated at 600 degrees C. for 3 hours. After strong heating, the sample was left to cool, and the mass of the residue was measured to the digit of 1 mg. The ash content was calculated according to Mathematical formula A below.

Ash content (residue of strong heating)(%)=residue/mass of sample×100 <Mathematical formula A>

<Seepage pH>

According to JIS K1474, a sample was weighed out in an amount of 1.0 g into a tall beaker, water (100 mL) was added to the resultant, and the resultant was heated for 5 minutes in a manner that boiling would continue silently. The resultant was cooled to room temperature (25 degrees C.), water was added to adjust the total amount to 100 mL, and the resultant was stirred well. The pH of the resultant was measured with a pH meter (obtained from HORIBA, Ltd., D-51).

### Porous Carbon Material Production Example 1

### <Production of Chaff-Derived Porous Carbon Material 1>

Chaff made in Akita Prefecture was used as a raw material.

The chaff was heated under a nitrogen current at 600 degrees C. for 5 hours, to obtain a carbide.

Next, the carbide was coarsely ground to a size of about 2 mm, subsequently immersed in a 1 mol % sodium hydroxide aqueous solution for removal of any ash content, and subsequently washed.

Next, the resultant was heated under a water vapor atmosphere at 950 degrees C. for 3.5 hours for activation, to obtain a chaff-derived porous carbon material 1.

### <Japanese Red Pine-Derived Porous Carbon Material 2>

INA RED PINE CHARCOAL (obtained from Sumi_Plus Co., Ltd.) was prepared as a porous carbon material 2.

### <Coconut Shell A-Derived Porous Carbon Material 3>

KURARAYCOAL GW (obtained from Kuraray Co., Ltd.) (for a water purifier) was prepared as a porous carbon material 3.

### <Coconut Shell B-Derived Porous Carbon Material 4>

FUNCTIONAL ACTIVATED CARBON FROM COCONUT SHELL (obtained from Sumi_Plus Co., Ltd.) was prepared as a porous carbon material 4.

### <Bamboo-Derived Porous Carbon Material 5>

A bamboo-derived porous carbon material (obtained from Takenosato LLC., product name: EDIBLE BAMBOO CHARCOAL POWDER) was prepared as a porous carbon material 5.

### <Broadleaf Tree-Derived Porous Carbon Material 6>

A broadleaf tree-derived porous carbon material (obtained from Kannabe Hakutan Kobo K.K., product name: KANNABE BLACK) (a plant charcoal powder pigment) was prepared as a porous carbon material 6.

Various property values of the porous carbon materials 1 to 6 are presented in Table 1 below.

TABLE 1

| | | | | | Specific | | | Bulk |
|---|---|---|---|---|---|---|---|---|
| | | Mesopore | Micropore | Median | surface | Ash | | specific |
| Porous carbon | Raw | volume | volume | diameter | area | content | Seepage | gravity |
| material No. | material | (cm³/g) | (cm³/g) | (micrometer) | (m²/g) | (%) | pH | (g/cm³) |
| Porous carbon | Chaff | 0.42 | 0.36 | 82 | 855 | 4.7 | 7.3 | 0.22 |
| material 1 | | | | | | | | |
| Porous carbon | Japanese | 0.09 | 0.21 | 9 | 495 | — | — | 0.30 |
| material 2 | red pine | | | | | | | |
| Porous carbon | Coconut | 0.07 | 0.55 | 90 | 1,167 | 0.2 | 6.7 | — |
| material 3 | shell A | | | | | | | |
| Porous carbon | Coconut | 0.12 | 0.66 | 19 | 1,615 | — | — | 0.29 |
| material 4 | shell B | | | | | | | |
| Porous carbon | Bamboo | — | — | 5.6 | — | 4.1 | 10.2 | 0.36 |
| material 5 | | | | | | | | |
| Porous carbon | Broadleaf | — | — | 9.7 | — | 4.6 | 10.0 | 0.38 |
| material 6 | tree | | | | | | | |

### Example 1

### <Adsorption Test on Advanced Glycation End Products (AGEs)>

(1) Alanine (25 g) and glucose (50 g) were dissolved in water (300 mL), and the resultant was heated at 95 degrees C. for 9 hours, cooled naturally, and subsequently diluted ten-fold, to produce an AGEs aqueous solution.

(2) The porous carbon materials 1 to 6 (0.3 g) were each added into the AGEs aqueous solution (40 mL), and the resultant was stirred for 5 minutes.

(3) The resultant was filtrated, the absorbance of the resultant was measured, and the rate of removal of advanced glycation end products was measured in the manner described below.

### [Absorbance Measuring Method]

The absorbance was measured with a visible spectrophotometer (obtained from JANWAY, 6300) using a cell having an optical path length of 10 mm.

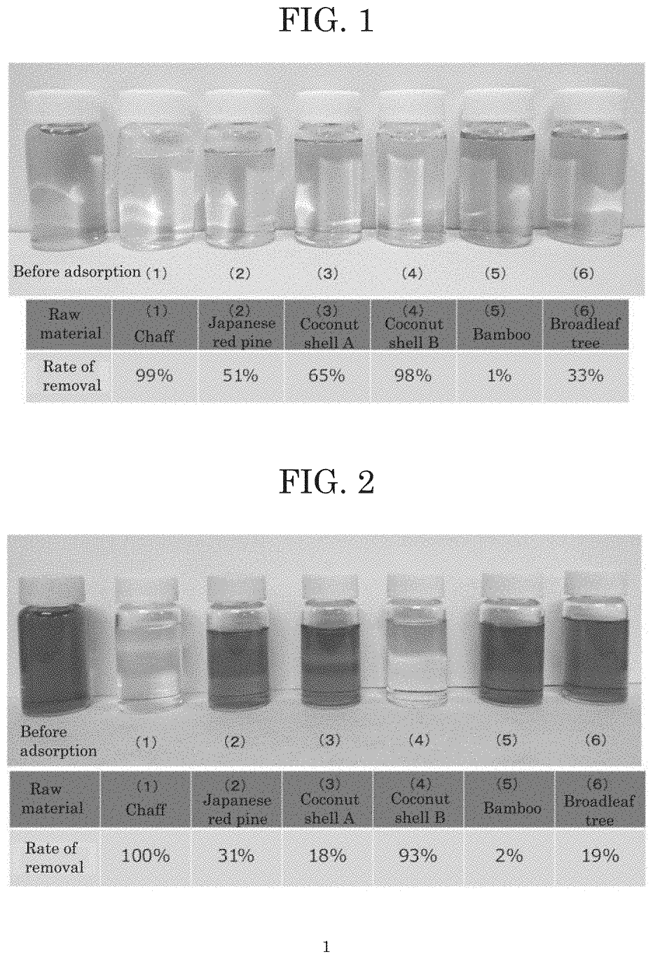

The absorbance to be measured was measured at a wavelength near the maximum absorption wavelength (315 nm) of the advanced glycation end products previously obtained. The results are presented in FIG. 1 and Table 1.

The rate of removal of the advanced glycation end products was calculated according to Mathematical formula 1 below.

Rate of removal of advanced glycation end products (%)=[(A−B)/A]×100 <Mathematical formula 1>

“A” represents the absorbance of the aqueous solution before treatment, and “B” represents the absorbance of the aqueous solution after treatment.

TABLE 1

| | | | | | Rate of |
|---|---|---|---|---|---|
| | | | | | removal |
| | | Mesopore | Micropore | Median | of |
| Porous carbon | Raw | volume | volume | diameter | AGEs |
| material No. | material | (cm³/g) | (cm³/g) | (micrometer) | (%) |
| Porous carbon | Chaff | 0.42 | 0.36 | 82 | 99 |
| material 1 | | | | | |
| Porous carbon | Japanese | 0.09 | 0.21 | 9 | 51 |
| material 2 | red pine | | | | |
| Porous carbon | Coconut | 0.07 | 0.55 | 90 | 65 |
| material 3 | shell A | | | | |
| Porous carbon | Coconut | 0.12 | 0.66 | 19 | 98 |
| material 4 | shell B | | | | |
| Porous carbon | Bamboo | — | — | 5.6 | 1 |
| material 5 | | | | | |
| Porous carbon | Broadleaf | — | — | 9.7 | 33 |
| material 6 | tree | | | | |

From the results of FIG. 1 and Table 1, the chaff-derived porous carbon material 1 and the coconut shell B-derived porous carbon material 4, both of which had a mesopore volume of 0.10 cm³/g or greater, were found to have an excellent adsorption effect represented by a rate of removal of advanced glycation end products (AGEs) of 90% or higher, and to be able to efficiently remove advanced glycation end products (AGEs), which were harmful-to-health substances.

### Example 2

### <Adsorption Test on Lipid (Chili Oil)>

(1) Water (20 g) and a chili oil (obtained from S&B Foods Inc.) (5 g) serving as a lipid were put into a cup.

(2) Each porous carbon material was added into the resultant in a predetermined amount. (For volume matching, the chaff-derived porous carbon material, which had a low bulk specific gravity, was added in 3 g, and the porous carbon materials derived from coconut shell A, coconut shell B, and Japanese red pine were each added in 5 g.)

(3) The total weight was measured.

(4) Five minutes later, any dry (non-water or chili oil-adsorbed) porous carbon material in the cup was removed by suctioning.

(5) The weight of the porous carbon material removed by suctioning was measured.

(6) The water and the chili oil were sucked up with a syringe, to measure the weight thereof.

(7) The total amount of the remaining cup, porous carbon material, and chili oil adsorbed to the porous carbon material was measured.

(8) The weights of the cup and the porous carbon material before the test were subtracted from the total amount obtained in (7), to obtain the adsorption amount of the chili oil.

(9) The amount of the chili oil adsorbed per 1 g of the porous carbon material was calculated based on the result of (8) and the addition amount of the porous carbon material.

TABLE 2

| | | | | | Amount |
|---|---|---|---|---|---|
| | | | | | (g) of |
| | | | | | chili oil |
| | | | | | adsorbed |
| | | | | | per 1 |
| | | | | | g of |
| | | Mesopore | Micropore | Median | porous |
| Porous carbon | Raw | volume | volume | diameter | carbon |
| material No. | material | (cm³/g) | (cm³/g) | (micrometer) | material |
| Porous carbon | Chaff | 0.42 | 0.36 | 82 | 1.8 |
| material 1 | | | | | |
| Porous carbon | Japanese | 0.09 | 0.21 | 9 | 0.7 |
| material 2 | red pine | | | | |
| Porous carbon | Coconut | 0.07 | 0.55 | 90 | 0.4 |
| material 3 | shell A | | | | |
| Porous carbon | Coconut | 0.12 | 0.66 | 19 | 0.9 |
| material 3 | shell B | | | | |

From the results of Table 2, the chaff-derived porous carbon material 1 was found to have a high adsorption amount of the lipid (chili oil) of 1.8 g, and to be able to quickly remove the lipid (chili oil), which was a harmful-to-health substance.

### Example 3

### <Adsorption Test on Histamine>

An adsorption test on histamine was performed and the rate of removal of histamine was obtained in the manner described below.

(1) A histamine aqueous solution was produced by adding histamine (100 mg) into water (500 mL).

(2) Each porous carbon material (0.3 g) was added into the histamine aqueous solution (40 mL), and the resultant was stirred for 5 minutes.

(3) The histamine aqueous solution subjected to filtration was caused to develop a color using a commercially available histamine quantification kit (product name “CHECKCOLOR HISTAMINE”, obtained from Kikkoman Corporation), and the absorbance of the histamine aqueous solution at a wavelength of 473 nm was measured with a visible spectrophotometer (obtained from JANWAY, 6300).

(4) The histamine concentration in the histamine aqueous solution was calculated in the manner specified in the histamine quantification kit.

(5) Based on the histamine concentrations in the histamine aqueous solution before and after removal, the rate of removal of histamine was calculated according to Mathematical formula 2 below.

Rate of removal of histamine (%)=[(A−B)/A]×100 <Mathematical formula 2>

“A” represents the histamine concentration in the histamine aqueous solution before treatment, and “B” represents the histamine concentration in the histamine aqueous solution after treatment.

TABLE 3

| | | | | | Rate of |
|---|---|---|---|---|---|
| | | | | Median | removal |
| | | Mesopore | Micropore | diameter | of |
| Porous carbon | Raw | volume | volume | (micro- | histamine |
| material No. | material | (cm³/g) | (cm³/g) | meter) | (%) |
| Porous carbon | Chaff | 0.42 | 0.36 | 82 | 91 |
| material 1 | | | | | |
| Porous carbon | Japanese | 0.09 | 0.21 | 9 | 27 |
| material 2 | red pine | | | | |
| Porous carbon | Coconut | 0.07 | 0.55 | 90 | 95 |
| material 3 | shell A | | | | |
| Porous carbon | Coconut | 0.12 | 0.66 | 19 | 95 |
| material 4 | shell B | | | | |
| Porous carbon | Bamboo | — | — | 5.6 | 21 |
| material 5 | | | | | |
| Porous carbon | Broadleaf | — | — | 9.7 | 20 |
| material 6 | tree | | | | |

From the results of Table 3, the chaff-derived porous carbon material 1, the coconut shell A-derived porous carbon material 3, and the coconut shell B-derived porous carbon material 4 were found to have an excellent histamine adsorption effect represented by a rate of removal of histamine of 90% or higher, and to be able to efficiently remove histamine, which was a harmful-to-health substance.

### Example 4

### <Adsorption Test on Edible Tar Dyes>

(1) Red No. 102 (0.1 g), blue No. 1 (0.1 g), and yellow No. 4 (0.1 g) each serving as an edible tar dye were each added into water (300 mL), to produce colored aqueous solutions of the respective dyes.

(2) Each porous carbon material (0.3 g) was added into each colored aqueous solution (40 mL), and the resultant was stirred for 5 minutes.

(3) The resultant was filtrated, the absorbance of the resultant was is measured, and the decoloration rate of each colored aqueous solution was measured in the manner descried below.

### [Absorbance Measuring Method]

The absorbance was measured with a visible spectrophotometer (obtained from JANWAY, instrument No. 6300) using a cell having an optical path length of 10 mm.

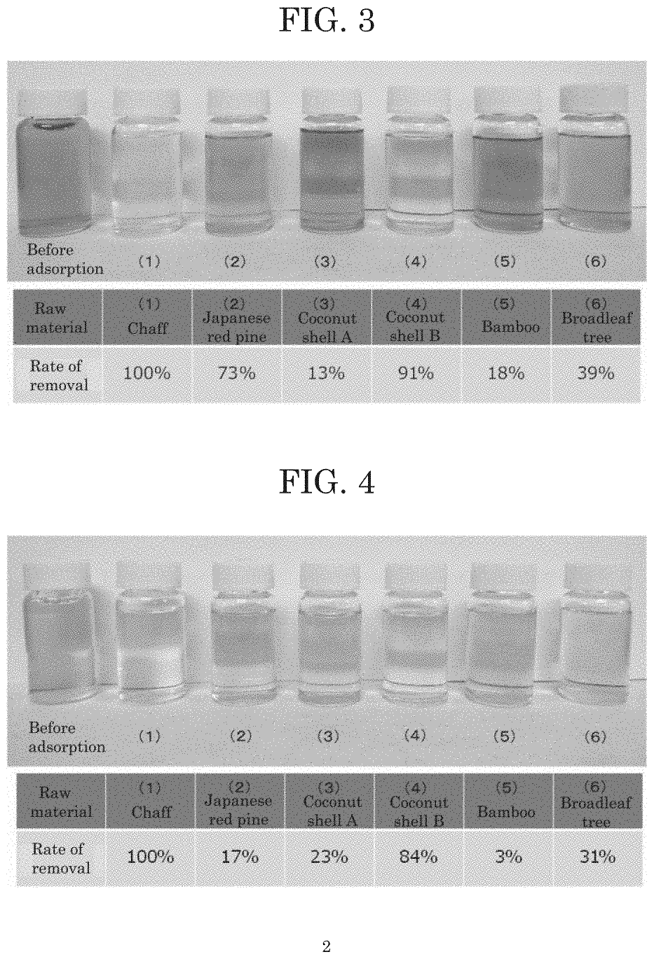

The absorbance to be measured was measured at a wavelength near the maximum absorption wavelengths (red No. 102: 510 nm, blue No. 1: 630 nm, and yellow No. 4: 430 nm) of the respective colored aqueous solutions previously obtained. The results are presented in FIG. 2 to FIG. 4 and Table 4.

The rate of removal of the edible tar dye was calculated according to Mathematical formula 3 below.

Rate of removal of edible tar dye (%)=[(A−B)/A]×100 <Mathematical formula 3>

“A” represents the absorbance of the colored aqueous solution before treatment, and “B” represents the absorbance of the colored aqueous solution after treatment.

TABLE 4

| | | | | | Rate of | Rate of | Rate of |
|---|---|---|---|---|---|---|---|
| | | Mesopore | Micropore | Median | removal of | removal of | removal of |
| Porous carbon | Raw | volume | volume | diameter | red No. 102 | blue No. 1 | yellow No. 4 |
| material No. | material | (cm³/g) | (cm³/g) | (micrometer) | (%) | (%) | (%) |
| Porous carbon | Chaff | 0.42 | 0.36 | 82 | 100 | 100 | 100 |
| material 1 | | | | | | | |
| Porous carbon | Japanese | 0.09 | 0.21 | 9 | 31 | 73 | 17 |
| material 2 | red pine | | | | | | |
| Porous carbon | Coconut | 0.07 | 0.55 | 90 | 18 | 13 | 23 |
| material 3 | shell A | | | | | | |
| Porous carbon | Coconut | 0.12 | 0.66 | 19 | 93 | 91 | 84 |
| material 4 | shell B | | | | | | |
| Porous carbon | Bamboo | — | — | 5.6 | 2 | 18 | 3 |
| material 5 | | | | | | | |
| Porous carbon | Broadleaf | — | — | 9.7 | 19 | 39 | 31 |
| material 6 | tree | | | | | | |

From the results of FIG. 2 to FIG. 4 and Table 4, the chaff-derived porous is carbon material 1 and the coconut shell B-derived porous carbon material 4, both of which had a mesopore volume of 0.10 cm³/g or greater, were found to have an excellent adsorption effect on the edible tar dyes (red No. 102, blue No. 1, and yellow No. 4), and to be able to efficiently remove the edible tar dyes. Particularly, the chaff-derived porous carbon material 1 achieved rates of removal of 100%, and was found to have an extremely high adsorbability.

### INDUSTRIAL APPLICABILITY

The harmful-to-health substance removing agent of the present invention contains many mesopores (i.e., has a high mesopore volume). Therefore, the harmful-to-health substance removing agent has a high adsorption amount of a harmful-to-health substance and a high adsorption speed of a harmful-to-health substance, and can efficiently remove a harmful-to-health substance even when ingested in a small amount. Accordingly, the harmful-to-health substance removing agent is applicable to removal of various harmful-to-health substances such as advanced glycation end products (AGEs), histamine, lipids, and edible tar dyes.

1: A harmful-to-health substance removing agent, comprising:
a plant-derived porous carbon material having a mesopore volume of 0.10 cm³/g or greater.

2: The harmful-to-health substance removing agent according to claim 1, wherein the mesopore volume of the plant-derived porous carbon material is 0.15 cm³/g or greater.

3: The harmful-to-health substance removing agent according to claim 1, wherein the mesopore volume of the plant-derived porous carbon material is greater than a micropore volume.

4: The harmful-to-health substance removing agent according to claim 1, wherein the plant-derived porous carbon material has a median diameter of 1 micrometer or greater but 200 micrometers or less.

5: The harmful-to-health substance removing agent according to claim 1, wherein a raw material of the plant-derived porous carbon material is chaff of rice, barley, wheat, rye, Japanese barnyard millet, or millet.

6: The harmful-to-health substance removing agent according to claim 5, wherein the raw material of the plant-derived porous carbon material is chaff of rice.

7: The harmful-to-health substance removing agent according to claim 1, wherein a harmful-to-health substance is an advanced glycation end product.

8: The harmful-to-health substance removing agent according to claim 7, wherein a rate of removal of the advanced glycation end product is 90% or higher.

9: The harmful-to-health substance removing agent according to claim 1, wherein a harmful-to-health substance is histamine.

10: The harmful-to-health substance removing agent according to claim 9, wherein a rate of removal of the histamine is 90% or higher.

11: The harmful-to-health substance removing agent according to claim 1, wherein a harmful-to-health substance is a lipid.

12: The harmful-to-health substance removing agent according to claim 11, wherein an amount of the lipid adsorbed per 1 g of the plant-derived porous carbon material is 1.0 g or greater.

13: The harmful-to-health substance removing agent according to claim 1, wherein a harmful-to-health substance is an edible tar dye.

14: The harmful-to-health substance removing agent according to claim 13, wherein a rate of removal of the edible tar dye is 90% or higher.

15: A health food, comprising the harmful-to-health substance removing agent according to claim 1.
